(19) United States
(12) Patent Application Publication
(10) Pub. No.: US 2017/0335365 A1
(43) Pub. Date: Nov. 23, 2017

(54) METHOD FOR DETECTING BACTERIA

(71) Applicant: NEC Solution Innovators, Ltd., Tokyo (JP)

(72) Inventors: Yoshihito YOSHIDA, Tokyo (JP); Iwao WAGA, Tokyo (JP)

(73) Assignee: NEC Solution Innovators, Ltd., Tokyo (JP)

(21) Appl. No.: 15/523,920

(22) PCT Filed: Jul. 23, 2015
(86) PCT No.: PCT/JP2015/071026

§ 371 Date: May 2, 2017

(30) Foreign Application Priority Data
Nov. 6, 2014 (JP) 2014-225682

(51) Int. Cl. C12Q 1/04; C12Q 1/02; C12Q 1/24

(52) U.S. Cl. CPC C12Q 1/04; C12Q 1/24; C12Q 1/02; C12Q 1/045

(57) ABSTRACT

The present invention provides a novel method for more simply and rapidly detecting target bacterial cells by a device using a binding molecule capable of binding to the target bacterial cells. In the method, a sample is incubated in a reagent for concentration of bacteria to cause the sample to react with a fluorescently-labeled binding molecule, and a fluorescence polarization degree is then detect to detect the target, and this is performed using a bacterial detection tool. The bacterial detection tool is obtained by attaching a sample collection tool to a main body. The sample collection tool includes a collection section and a connection section that is connected to the main body. The main body includes a reagent storage chamber, a collection section storage chamber, and a connection section that is connected to the sample collection tool. The reagent storage chamber and the collection section storage chamber are separated from each other. The sample collection tool and the main body are connected to each other at the connection section of the sample collection tool and the connection section of the main body after the preparation step and before the incubation step with the collection section being placed inside the collection section storage chamber. The reagent storage chamber and the collection section storage chamber internally communicate with each other after the preparation step to perform the incubation step and the reaction step.
h a sample is collected; and
   a connection section that is connected to the main body,
   the main body comprises:
   a reagent storage chamber that contains the reagent and the fluorescently-labeled binding molecule;
   a collection section storage chamber that contains the collection section of the sample collection tool; and
   a connection section that is connected to the sample collection tool,
   the reagent storage chamber and the collection section storage chamber are separated from each other, and
   the sample collection tool and the main body are connected to each other at the connection section of the sample collection tool and the connection section of the main body with the collection section of the sample collection tool being placed inside the collection section storage chamber of the main body.

### TECHNICAL FIELD

The present invention relates to a method for detecting bacteria

### BACKGROUND ART

Food poisonings caused by infections with microbes such as Escherichia coli and Salmonella enterica have been increased in recent years. A major cause of this problem is washing failures. For example, food-processing factories are thus required to detect bacterial cells in an inspection.

A method for detecting bacterial cells is, for example, a method using a swabbing device having a rod and a swab at the tip of the rod (e.g., Patent Documents 1 and 2). In this method, a sample is collected at the tip of the device, the tip is immersed in a medium to cultivate the sample, a substrate to an enzyme for target bacterial cells are further added as a reagent, and the enzyme reaction is detected to indirectly detect the target bacterial cells.

However, for the detection of a reaction between the enzyme and the substrate, target bacterial cells may not be specifically detected for the reason that various bacterial cells grown in an cultivation step cause reactions between enzymes and the substrates derived from the various bacterial cells. The target bacterial cells are thus required to be selectively cultivated in the cultivation step, and the cultivation requires time and effort.

Such problem can be solved by, for example, employing the ELISA method and the SPR method using a binding molecule (e.g., an antibody, an aptamer, a binding peptide, a carbohydrate chain, a binding protein) that specifically binds to the target bacterial cells. However, these methods require a washing step to remove the binding molecule binding to no target bacterial cells after causing the target bacterial cells and the binding molecule to be bonded to each other. These methods thus cannot be performed in the swabbing device that is not based on the washing step.

### PRIOR ART DOCUMENTS

### Patent Documents

Patent Document 1: Japanese Patent No. 3431812
Patent Document 2: JP 2013-516179 A

### SUMMARY OF INVENTION

### Problem to be Solved by the Invention

Hence, the present invention is intended to provide a novel method for more simply and rapidly detecting target bacterial cells by a device using a binding molecule capable of binding to the target bacterial cells.

### Means for Solving Problem

The present invention provides a method for detecting a bacterium including:

a preparation step of preparing a sample;

an incubation step of incubating the sample in a reagent for concentration of bacteria;

a reaction step of causing the sample in the reagent and a fluorescently-labeled binding molecule to be bonded to a target bacterium to react with each other; and

a detection step of detecting a fluorescence polarization degree of the fluorescently-labeled binding molecule to detect the target bacterium, wherein

the preparation step is performed using a sample collection tool,

the incubation step, the reaction step, and the detection step are performed using a bacterial detection tool obtained by attaching the sample collection tool to a main body,

the sample collection tool includes:
a collection section in which the sample is collected; and
a connection section that is connected to the main body,

the main body includes:
a reagent storage chamber that contains the reagent and the fluorescently-labeled binding molecule;
a collection section storage chamber that contains the collection section of the sample collection tool; and
a connection section that is connected to the sample collection tool,

the reagent storage chamber and the collection section storage chamber are separated from each other,

the sample collection tool and the main body are connected to each other at the connection section of the sample collection tool and the connection section of the main body after the preparation step and before the incubation step with the collection section of the sample collection tool being placed inside the collection section storage chamber of the main body, and

the reagent storage chamber and the collection section storage chamber internally communicate with each other after the preparation step to perform the incubation step and the reaction step.

The present invention provides a bacterial detection tool for use in the method of the present invention, the bacterial detection tool including:

a sample collection tool; and

a main body, wherein

the sample collection tool includes:
a collection section in which a sample is collected; and
a connection section that is connected to the main body,

the main body includes:
a reagent storage chamber that contains the reagent and the fluorescently-labeled binding molecule;
a collection section storage chamber that contains the collection section of the sample collection tool; and
a connection section that is connected to the sample collection tool,

the reagent storage chamber and the collection section storage chamber are separated from each other, and

the sample collection tool and the main body are connected to each other at the connection section of the sample collection tool and the connection section of the main body with the collection section of the sample collection tool being placed inside the collection section storage chamber of the main body.

### Effects of the Invention

The present invention uses a fluorescently-labeled binding molecule as a binding molecule that binds to a target bacteria and detects the fluorescence polarization degree of the fluorescently-labeled binding molecule varied according to the binding state between the fluorescently-labeled binding molecule and the target to detect the target. This method does not require the above-mentioned washing step. The method of the present invention thus can simply and promptly detect the target using a bacterial detection tool including a sample collection tool and a main body. The method of the present invention is therefore really useful in the fields of, for example, food manufacturing, food management, and food distribution.

### BRIEF DESCRIPTION OF DRAWINGS

FIGS. 1A and 1B show an example of the bacterial detection tool according to the present invention. FIG. 1A is a sectional view of the bacterial detection tool, and FIG. 1B is a sectional view showing the state of the bacterial detection tool in use.

FIGS. 2A, 2B, and 2C show other examples of the bacterial detection tool according to the present invention. FIGS. 2A, 2B, and 2C are sectional views showing variations of the bacterial detection tool.

FIGS. 3A, 3B, 3C, 3D, and 3E show still other examples of the bacterial detection tool according to the present invention. FIGS. 3A, 3B, 3C, 3D, and 3E are sectional views showing variations of the bacterial detection tool.

FIG. 4 is a sectional view showing another example of the bacterial detection tool according to the present invention.

FIGS. 5A, 5B, and 5C show still other examples of the bacterial detection tool according to the present invention. FIGS. 5A, 5B, and 5C are sectional views showing variations of the bacterial detection tool.

FIG. 6 is a sectional view showing still another example of the bacterial detection tool according to the present invention.

FIGS. 7A, 7B, and 7C show still other examples of the bacterial detection tool according to the present invention. FIGS. 7A, 7B, and 7C are sectional views showing variations of the bacterial detection tool.

FIG. 8 is a sectional view showing still another example of the bacterial detection tool according to the present invention.

FIGS. 9A, 9B, and 9C show still other examples of the bacterial detection tool according to the present invention. FIGS. 9A, 9B, and 9C are sectional views showing variations of the bacterial detection tool.

### DESCRIPTION OF EMBODIMENTS

For example, in the method of the present invention, the reagent storage chamber contains a mixed reagent of the reagent and the fluorescently-labeled binding molecule, and the reagent storage chamber and the collection section storage chamber internally communicate with each other after the preparation step to bring the sample in the collection section of the reagent collection tool and the mixed reagent into contact with each other and perform the incubation step and the reaction step in parallel.

For example, in the method of the present invention, the reagent storage chamber of the main body in use of the bacterial detection tool is disposed above or below the collection section storage chamber.

For example, in the method of the present invention, the reagent storage chamber separately includes: a first reagent storage chamber; and a second reagent storage chamber, the first reagent storage chamber contains the reagent, the second reagent storage chamber contains the fluorescently-labeled binding molecule, the first reagent storage chamber and the second reagent storage chamber are separated from the collection section storage chamber, the first reagent storage chamber and the collection section storage chamber internally communicate with each other after the preparation step to bring the sample in the collection section of the sample collection tool and the reagent into contact with each other and perform the incubation step, and the second reagent storage chamber and the collection section storage chamber internally communicate with each other after the incubation step to bring the incubated sample and the fluorescently-labeled binding molecule into contact with each other and perform the reaction step.

For example, in the method of the present invention, the first reagent storage chamber and the second reagent storage chamber in the main body in use of the bacterial detection tool are both disposed above or below the collection section storage chamber.

For example, in the method of the present invention, either one of the first reagent storage chamber and the second reagent storage chamber in the main body in use of the bacterial detection tool is disposed above the collection section storage chamber, and the other is disposed below the collection section storage chamber.

For example, the method of the present invention further includes a disinfection step of treating the incubated sample with a disinfectant after the detection step, the main body further includes: a disinfectant storage chamber that contains the disinfectant, the reagent storage chamber and the collection section storage chamber are separated from the disinfectant storage chamber, and the disinfectant storage chamber and the collection section storage chamber internally communicate with each other after the detection step to perform the disinfection step.

For example, in the method of the present invention, the disinfectant storage chamber in the main body in use of the bacterial detection tool is disposed above or below the collection section storage chamber.

For example, in the method of the present invention, the collection section storage chamber serves as a reaction chamber in which the incubation step and the reaction step are performed and further includes a detection region, the detection region is formed of a member capable of detecting a fluorescence polarization degree in the collection section storage chamber from outside, the reagent storage chamber contains a mixed reagent of the reagent and the fluorescently-labeled binding molecule, and the reagent storage chamber and the collection section storage chamber internally communicate with each other after the preparation step to introduce the mixed reagent from the reagent storage chamber to the collection section storage chamber and perform the incubation step and the reaction step in parallel.

For example, in the method of the present invention, the collection section storage chamber serves as a reaction chamber in which the incubation step and the reaction step are performed and further includes a detection region, the detection region is formed of a member capable of detecting a fluorescence polarization degree in the collection section storage chamber from outside, the reagent storage chamber separately includes: a first reagent storage chamber; and a second reagent storage chamber, the first reagent storage chamber contains the reagent, the second reagent storage chamber contains the fluorescently-labeled binding molecule, the first reagent storage chamber and the second reagent storage chamber are separated from the collection section storage chamber, the first reagent storage chamber and the collection section storage chamber internally communicate with each other after the preparation step to introduce the reagent from the first reagent storage chamber to the collection section storage chamber and perform the incubation step, and the second reagent storage chamber and the collection section storage chamber internally communicate with each other after the incubation step to introduce the fluorescently-labeled binding molecule from the second reagent storage chamber to the collection section storage chamber and perform the reaction step.

For example, the method of the present invention further includes a disinfection step of treating the incubated sample with a disinfectant after the detection step, the main body further includes a disinfectant storage chamber that contains the disinfectant, the reagent storage chamber and the collection section storage chamber are separated from the disinfectant storage chamber, and the disinfectant storage chamber and the collection section storage chamber internally communicate with each other after the detection step to introduce the disinfectant from the disinfectant storage chamber to the collection section storage chamber and perform the disinfection step.

For example, in the method of the present invention, the storage chambers to be paired and communicate with each other in the main body are connected to each other by inserting a hollow tube for communication, the hollow tube includes a closed portion at one end, the storage chambers do not internally communicate with each other by the closed portion of the hollow tube, and the hollow tube is cut at a certain position between both ends in the axial direction to open the both ends of the hollow tube and to cause the storage chambers to internally communicate with each other.

For example, in the method of the present invention, the main body further includes a through hole forming unit, either one of the storage chambers to be paired and communicate with each other in the main body includes an opening capable of communicating with the other storage chamber, and a trough hole is formed in the other chamber by the through hole forming unit to cause the storage chambers to internally communicate with each other through the opening in the one storage chamber and the through hole formed in the other storage chamber.

For example, in the method of the present invention, the reagent storage chamber in the main body is a breakable capsule, and the capsule is broken to cause the reagent storage chamber and the collection section storage chamber to internally communicate with each other.

For example, in the method of the present invention, the reagent storage chamber serves as a reaction chamber in which the incubation step and the reaction step are performed, contains a mixed reagent of the reagent and the fluorescently-labeled binding molecule, and further includes a detection region, the detection region is formed of a member capable of detecting a fluorescence polarization degree in the collection section storage chamber from outside, and the collection section placed inside the collection section storage chamber is placed inside the reagent storage chamber after the preparation step to bring the mixed reagent in the reagent storage chamber into contact with the collection section and perform the incubation step and the reaction step in parallel.

For example, in the method of the present invention, the reagent storage chamber separately includes a first reagent storage chamber and a second reagent storage chamber, the first reagent storage chamber contains the reagent, the second reagent storage chamber contains the fluorescently-labeled binding molecule, the first reagent storage chamber and the second reagent storage chamber are separated from the collection section storage chamber, the first reagent storage chamber serves as a reaction chamber in which the incubation step is performed, the second reagent storage chamber serves as a reaction chamber in which the reaction step is performed and further includes a detection region, the detection region is formed of a member capable of detecting a fluorescence polarization degree in the second reagent storage chamber from outside, the collection section placed inside the collection section storage chamber is placed inside the first reagent storage chamber after the preparation step to bring the collection section into contact with the reagent in the first reagent storage chamber and perform the incubation step, and the collection section placed inside the first reagent storage chamber is placed inside the second reagent storage chamber after the incubation step to introduce a bacteria-concentrated reagent after the incubation into the second reagent storage chamber and perform the reaction step.

For example, the method of the present invention further includes a disinfection step of treating the incubated sample with a disinfectant after the detection step, the main body further includes a disinfectant storage chamber that contains the disinfectant, the reagent storage chamber and the collection section storage chamber are separated from the disinfectant storage chamber, and the disinfectant storage chamber and the collection section storage chamber internally communicate with each other after the detection step to introduce the disinfectant from the disinfectant storage chamber to the collection section storage chamber and perform the disinfection step.

For example, in the method of the present invention, the reagent is a medium for the target bacterium.

For example, in the method of the present invention, a binding molecule to be bonded to the target bacterium in the fluorescently-labeled binding molecule is at least one selected from the group consisting of an aptamer, a low-molecular-weight compound, a carbohydrate chain, a peptide, a protein, a nucleic acid, virus, and phage.

For example, in the method of the present invention, the protein is an antibody.

(1. Method for Detecting Bacteria)

The following describes the method of the present invention in detail.

As mentioned above, the method of the present invention includes:

a preparation step of preparing a sample;

an incubation step of incubating the sample in a reagent for concentration of bacteria;

a reaction step of causing the sample in the reagent and a fluorescently-labeled binding molecule to be bonded to a target bacterium to react with each other; and

a detection step of detecting a fluorescence polarization degree of the fluorescently-labeled binding molecule to detect the target bacterium, wherein

the preparation step is performed using a sample collection tool,

the incubation step, the reaction step, and the detection step are performed using a bacterial detection tool obtained by attaching the sample collection tool to a main body,

the sample collection tool includes:
a collection section in which the sample is collected; and
a connection section that is connected to the main body,

the main body includes:
a reagent storage chamber that contains the reagent and the fluorescently-labeled binding molecule;
a collection section storage chamber that contains the collection section of the sample collection tool; and
a connection section that is connected to the sample collection tool,

the reagent storage chamber and the collection section storage chamber are separated from each other,

the sample collection tool and the main body are connected to each other at the connection section of the sample collection tool and the connection section of the main body after the preparation step and before the incubation step with the collection section of the sample collection tool being placed inside the collection section storage chamber of the main body, and

the reagent storage chamber and the collection section storage chamber internally communicate with each other after the preparation step to perform the incubation step and the reaction step.

The detection of the target by the detection of a fluorescence polarization degree in the present invention is, for example, based on the fluorescence polarization. The fluorescence polarization is a measurement based on the characteristic that, in irradiation of a labeling substance with polarized excitation light, the polarization degree of the fluorescence emitted from the labeling substance generally differs according to the molecular weight of the molecule labeled with the labeling substance. The fluorescently-labeled binding molecule obtained by labeling the binding molecule with the fluorescently-labelling substance is used in the present invention. The binding between the target and the fluorescently-labeled binding molecule thus can be detected by the fluorescence polarization. Specifically comparing the state of the fluorescently-labeled binding molecule binding to no target and the state of the fluorescently-labeled binding molecule binding to the target, the former has a relatively small molecular weight and thus shows a relatively low fluorescence polarization degree, whereas the latter has a relatively large molecular weight and thus shows a relatively high fluorescence polarization degree. The binding between the target and the fluorescently-labeled binding degree thus can be detected by, for example, comparing the fluorescence polarization degree of the fluorescently-labeled binding molecule binding to no sample and that of the fluorescently-labeled bonding molecule binding to the sample. Alternatively, the binding between the target and the fluorescently-labeled binding molecule can be detected by evaluating the fluorescence polarization degree of the fluorescently-labeled binding molecule being in contact with the sample based on at least either one of the fluorescence polarization degree of the fluorescently-labeled binding molecule binding to no target and that of the fluorescently-labeled binding molecule binding to the target as an evaluation criteria.

The preparation step in the present invention is a step of preparing a sample. The kind of the sample is not limited to particular kinds in the present invention. Examples of the sample include a biological body-derived sample, a food and beverage-derived sample, and an environmental sample. The biological body is not limited to particular biological bodies, and examples thereof include humans; nonhuman mammals such as cattle, swine, sheep, mice, rats, rabbits, and horses; and animals such as birds and fish. Examples of the biological body-derived sample include feces, body fluids, skins, flesh, mucosa, and body hair. Examples of the food and beverage-derived sample include beverages, food, food ingredients. Examples of the environmental sample include organisms, water, the ground, and the atmosphere. Examples of the water as the sample include ground water, river water, seawater, and domestic wastewater. Examples of the environmental sample further include adherent substances in food-processing factories and kitchens.

The bacterium to be detected in the present invention are not limited to particular bacteria, and examples thereof include Listeria, Salmonella, pathogenic Escherichia coli, Staphylococcus aureus, Campylobacter, Vibrio, Clostridium perfringens, Vibrio cholerae, and Haemophilus influenza.

The target to be detected in the present invention is not limited to bacteria and may be a bacterium-derived substance. For the bacterium-derived substance that can specify the bacterium as the target to be detected, the bacterium specified by the substance can be indirectly detected by detecting the bacterium-derived substance, for example. Examples of the bacterium-derived substance include a bacterium-derived secretion and an endogenous substance of the bacterium. In this case, for example, a binding molecule that binds to the bacterium-derived substance is preferably used in the present invention.

The incubation step in the present invention is a step of incubating the sample in the reagent for concentration of bacteria. The concentration of bacteria in the present invention can be, for example, concentration by cultivating and growing bacteria in a medium. The reagent for concentration of bacteria can be, for example, a medium for growing target bacterium. The kind of the medium is not limited to particular media and can be set, as appropriate, according to the target bacterium, for example. The target can be detected using a fluorescently-labeled binding molecule that binds to the target in the present invention. The medium thus may be any media widely used for various bacteria in addition to media that specifically grow the target, for example.

The conditions of the incubation step are not limited to particular conditions and can be determined, as appropriate, according to the kinds of the sample and the target. For the above-mentioned reason, the conditions under which the target is specifically grown are not limited in the present invention, for example.

The reaction step in the present invention is a step of causing the sample in the reagent and a fluorescently-labeled binding molecule to be bonded to the target bacterium to react with each other. In the present invention, the incubation step and the reaction step may be, for example, performed in parallel, or the reaction step may be performed after the incubation step. In the former case, the both steps may be performed using a mixed reagent obtained by mixing the reagent for concentration of bacteria and the fluorescently-labeled binding molecule, for example, or the both steps may be performed after bringing the reagent and the fluorescently-labeled binding molecule into contact with the sample in no particular order. In the latter case, the reagent and the sample may be brought into contact with each other to perform the incubation step, and the sample and the fluorescently-labeled binding molecule may thereafter be brought into contact with each other to perform the reaction step, for example. Hereinafter, the form of using the mixed reagent is also referred to as a “one-reagent system”, and the form of separately using the reagent and the fluorescently-labeled binding molecule is also referred to as a “two-reagent system”.

In the fluorescently-labeled binding molecule, a binding molecule that binds to the target is labeled with a fluorescent substance. The binding molecule is only required to be capable of binding to the target and is not limited to particular molecules and can be determined, as appropriate, according to the kind of the target.

Specific examples of the binding molecule include an aptamer, a low-molecular-weight compound, a carbohydrate chain, a peptide, a protein, a nucleic acid, virus, and phage. The protein can be, for example, an antibody. The aptamer is, for example, a nucleic acid molecule that binds to the target, examples thereof include RNA, DNA, chimera of RNA and DNA, and the aptamer may further include an artificial nucleic acid.

The fluorescent substance is not limited to particular substances, and examples thereof include pigments such as pyrene, TAMRA, fluorescein, a Cy3 pigment, a Cy5 pigment, a FAM pigment, a rhodamine pigment, a Texas Red pigment; and fluorophores such as JOE, MAX, HEX, and TYE. Specific examples of the pigments include Alexa pigments such as Alexa 488 and Alexa 647. For example, the fluorescent substance may directly be connected to the binding molecule or may indirectly be connected to the binding molecule via a linker.

For an aptamer as the binding molecule, the fluorescent substance may be bonded to any position of the aptamer, and the position can be at least either one of the 5′ end or the 3′ end. For example, for the fluorescent substance binding to the aptamer via the linker, the linker is not limited to particular linkers, and examples thereof include the above-mentioned nucleic acid molecules for the aptamer.

The conditions of the reaction step are not limited to particular conditions and can be determined, as appropriate, according to the kinds of the sample, the target, and the binding molecule. In the reaction step, the reaction temperature is, for example, from 4° C. to 37° C., from 18° C. to 25° C., and the reaction time is, for example, from 10 to 120 minutes, from 30 to 60 minutes.

The other components may be present together in the reaction step in addition to the fluorescently-labeled binding molecule, for example. Examples of the other components include water, aqueous solvents such as buffer solutions, a surfactant, a salt, a metal ion, and additives.

The detection step in the present invention is a step of detecting a fluorescence polarization degree of the fluorescently-labeled binding molecule to detect the target. As mentioned above, the fluorescence polarization degree of the fluorescently-labeled binding molecule binding to the target is higher than that of the fluorescently-labeled binding molecule binding to no target. The target thus can be detected by detecting the fluorescence polarization degree, and for example, the target can be analyzed qualitatively and quantitatively.

A method for detecting the fluorescence polarization degree in the detection step is not limited to particular methods and can be determined, as appropriate, according to the kind of the fluorescent substance, for example. In a specific example, for Alexa 647 as the labeling substance, the wavelength of the polarized excitation light is, for example, from 620 to 680 nm, the detection wavelength of the polarization degree is, for example, 660 to 800 nm. The time for irradiation of a labeling substance with the polarized excitation light is not limited to particular times and can be, for example, 1 to 5 nano seconds.

The method of the present invention may further include a disinfection step of treating the incubated sample with a disinfectant after the detection step. The disinfection step can kill bacteria grown by the incubation of the sample, avoid the environmental impacts of wastes, and improve the effectiveness.

The disinfectant (bactericide) in the disinfection step is not limited to particular disinfectants (bactericides), and a component that exhibits a disinfection (bactericidal) effect can be used. Examples of the disinfectant (bactericide) include sodium hypochlorite and chromium dioxide.

The method of the present invention is performed using the bacterial detection tool including the sample collection tool and the main body as mentioned above. Specifically, in the method of the present invention, the preparation step for the sample is performed using the sample collection tool, and the incubation step, the reaction step, and the detection step are performed using the bacterial detection tool obtained by attaching the sample collection tool to the main body. In the bacterial detection tool, the sample collection tool is detachable from the main body.

In the present invention, being above and being below are, for example, being above and being below in use of the bacterial detection tool. Specifically, the description is made assuming that the collection section side of the sample collection tool is located below the sample collection tool, the side opposite to the collection section of the sample collection tool (e.g., the connection section side) is located above the sample collection tool.

As mentioned above, the sample collection tool includes a collection section in which a sample is collected, and a connection section that is connected to the main body. In the present invention, the sample collection tool is, for example, a rod-shaped tool and includes the collection section at one tip of a rod-shaped support member, and the a connection section is located in a region above the support member (for example, at the upper end or a upper end portion of the support member). Examples of the sample collection tool include a swab, a brush, a spatula, and a spoon.

The collection section of the sample collection tool is not limited to particular sections and can be, for example, a ball of fiber, and examples of the fiber include cotton and a resin fiber. The connection section of the sample collection tool is not limited to particular sections and is only required to be capable of connecting to the connection section of the main body. The connection section of the sample collection tool is described below together with the connection section of the main body.

The preparation step of preparing a sample using a sample collection tool can be performed by, for example, rubbing a part to be detected with the collection section of the sample collection tool to collect a sample in the collection section. The part to be detected is not limited to particular parts and is, for example, a part in which the above-mentioned sample is present.

As mentioned above, the main body includes: a reagent storage chamber that contains the reagent for concentration of bacteria and the fluorescently-labeled binding molecule; a collection section storage chamber that contains the collection section of the sample collection tool; and a connection section that is connected to the sample collection tool, and the reagent storage chamber and the collection section storage chamber are separated from each other. In the method of the present invention, the sample collection tool and the main body are connected to each other at the connection section of the sample collection tool and the connection section of the main body after the preparation step and before the incubation step with the collection section of the sample collection tool being placed inside the collection section storage chamber of the main body, and the reagent storage chamber and the collection section storage chamber internally communicate with each other after the preparation step to perform the incubation step and the reaction step.

The reagent storage chamber in the main body may be, for example, constituted by one storage chamber or two or more separated storage chambers. In the present invention, “the reagent storage chamber separately including two or more storage chambers” may be, for example, in the form of including two or more separated chambers in one storage chamber or in the form of including two or more separated storage chambers as the reagent storage chamber, i.e., the form of separately forming two or more separated storage chambers.

As mentioned above, for the use of a mixed reagent of the regent for concentration of bacteria and the fluorescently-labeled binding molecule are used in the present invention, the reagent storage chamber is, for example, one storage chamber that contains the mixed reagent. For the separate use of the reagent for concentration of bacteria and the fluorescently-labeled binding molecule in the present invention, the latter state is preferable, and for example, the reagent storage chamber separately includes a first reagent storage chamber and a second reagent storage chamber, the first reagent storage chamber contains the reagent for concentration of bacteria, and the second reagent storage chamber contains the fluorescently-labeled binding molecule. In this case, the first reagent storage chamber and the second reagent storage chamber are, for example, separated from the collection section storage chamber.

The position of the reagent storage chamber to be disposed in the main body is not limited to particular positions, and for example, the regent storage chamber in use of the bacterial detection tool may be disposed above or below the collection section storage chamber. For the reagent storage chamber including two or more separated storage chambers as mentioned above, all of the storage chambers may be disposed above or below the collection section storage chamber, or alternatively, either one of the storage chambers is disposed above the collection section storage chamber, and the other storage chamber(s) is disposed below the collection section storage chamber, for example. For the main body including the first reagent storage chamber and the second reagent storage chamber, the former may be disposed above the collection section storage chamber and the latter may be disposed below the collection section storage chamber, or alternatively, the former may be disposed below the collection section storage chamber, and the latter may be disposed above the collection section storage chamber, for example.

As described above, for the main body including one or more storage chambers disposed the collection section storage chamber, this storage chamber(s) is hereinafter also collectively referred to as an “upper main body”, and the collection section storage chamber is hereinafter also referred to as a “lower main body”. For the main body including one or more storage chambers disposed below the collection section storage chamber, this storage chamber(s) is hereinafter also collectively referred to as a “lower main body”, and the collection section storage chamber is hereinafter also referred to as an “upper main body”. For the main body including one or more storage chambers disposed above the collection section storage chamber and one or more storage chambers disposed below the collection section storage chamber, the upper storage chamber(s) is hereinafter also collectively referred to as an “upper main body”, the collection section storage chamber is hereinafter also referred to as a “middle main body”, and the lower storage chamber(s) is hereinafter also collectively referred to as a “lower main body”.

For example, for the use of the mixed reagent in the present invention, the reagent storage chamber and the collection section storage chamber internally communicate with each other after the preparation step to bring the sample in the collection section of the sample collection tool and the mixed reagent into contact with each other, and the incubation step and the reaction step thus can be performed in parallel. For example, for the separate use of the reagent for concentration of bacteria and the fluorescently-labeled binding molecule in the present invention, the first reagent storage chamber and the collection section storage chamber internally communicate with each other after the preparation step to bring the sample in the collection section of the sample collection tool and the reagent for concentration of bacteria into contact with each other, and the incubation step thus can be performed, and the second reagent storage chamber and the collection section storage chamber then communicate with each other after the incubation step to bring the incubated sample and the fluorescently-labeled binding molecule into contact with each other, and the reaction step thus can be performed.

As mentioned above, for the further use of the disinfectant in the present invention, the main body further includes, for example, a disinfectant storage chamber that contains the disinfectant, and in this case, the reagent storage chamber and the collection section storage chamber are separated from the disinfectant storage chamber. As mentioned above, for the use of the mixed reagent in the present invention, the main body separately includes, for example, the reagent storage chamber that contains the mixed reagent and the disinfectant storage chamber. For the separate use of the reagent for concentration of bacteria and the fluorescently-labeled binding molecule, the main body separately includes, for example, the first reagent storage chamber, the second reagent storage chamber, and the disinfectant storage chamber.

The position of the disinfectant storage chamber to be disposed in the main body is not limited to particular positions in the present invention. For example, the disinfectant storage chamber in use of the bacterial detection tool may be disposed above or below the collection section storage chamber. The disinfectant storage chamber may be disposed on the same side as or the different side from the reagent storage chamber with respect to the collection section storage chamber.

For the use of the disinfectant in the present invention, the disinfectant storage chamber and the collection section storage chamber, for example, internally communicate with each other after the detection step, and the disinfection step thus can be performed.

The storage chambers separated from each other in the present invention is, for example, in the form of disposing the storage chambers to be separated from each other, in the form of having partitions for separating the storage chambers with the storage chambers being adjacent to each other but not being in communication with each other.

For example, in the method of the present invention, the collection section storage chamber or the reagent storage chamber may be a reaction chamber.

The form of the collection section storage chamber as a reaction chamber is described below.

For the one-reagent system using the mixed reagent, the collection section storage chamber serves as, for example, a reaction chamber in which the incubation step and the reaction step are performed. Furthermore, it is preferred that the collection section storage chamber further includes, for example, a detection region, and the detection region is formed of a member capable of detecting a fluorescence polarization degree in the collection section storage chamber from outside. In this form, the reagent storage chamber and the collection section storage chamber, for example, internally communicate with each other after the preparation step to introduce the mixed reagent from the reagent storage chamber to the collection section storage chamber. The incubation step and the reaction step thus can be performed in parallel.

For the two-reagent system separately using the reagent for concentration of bacteria and the fluorescently-labeled binding molecule, the collection section storage chamber serves as, for example, a reaction chamber in which the incubation step and the reaction step are performed. Furthermore, it is preferred that the collection section storage chamber further includes, for example, a detection region, and the detection region is formed of a member capable of detecting a fluorescence polarization degree in the collection section storage chamber from outside. In this form, the first reagent storage chamber and the collection section storage chamber, for example, internally communicate with each other after the preparation step to introduce the reagent for concentration of bacteria from the first reagent storage chamber to the collection section storage chamber. The incubation step thus can be performed. The second reagent storage chamber and the collection section storage chamber then internally communicate with each other after the incubation step to introduce the fluorescently-labeled binding molecule from the second reagent storage chamber to the collection section storage chamber The reaction step thus can be performed.

A member for forming the detection region is only required to be capable of detecting the fluorescence polarization degree from outside and is not limited to particular members. The member is, for example, a transparent member, and specific examples thereof include glass and a plastic. Examples of the plastic include polystyrene and polycarbonate. The same applies hereinafter.

For the method of the present invention further including the disinfection step in the form of the one-reagent system or the two-reagent system, the disinfectant storage chamber and the collection section storage chamber, for example, internally communicate with each other after the detection step to introduce the disinfectant from the disinfectant storage chamber to the collection section storage chamber, and the disinfection step thus can be performed.

For the collection section storage chamber as a reaction chamber, the method for communicating the storage chambers with each other is not limited to particular methods, and examples thereof include the following forms A, B, and C.

The form A is a method using a hollow tube. In this case, storage chambers to be paired and communicate with each other in the main body are connected to each other by placing a hollow tube for communication, and the hollow tube includes a closed portion at one end. The storage chambers thus does not internally communicate with each other by the closed portion of the hollow tube, and the hollow tube is cut at a certain position between both ends in the axial direction to open the both ends of the hollow tube, and the storage chamber thus can internally communicate with each other. Examples of the storage chambers to be paired include: the reagent storage chamber and the collection section storage chamber; the first reagent storage chamber and the collection section storage chamber; the second reagent storage chamber and the collection section storage chamber; and the disinfectant storage chamber and the collection section storage chamber (the same applies hereinafter).

The form B is a method using a through hole forming unit. In this case, the main body further includes a through hole forming unit, and either one of the storage chambers to be paired and communicate with each other in the main body includes an opening capable of communicating with the other storage chamber, and the other storage chamber is capable of forming an opening by the through hole forming unit. A through hole is then formed in the other storage chamber by the through hole forming unit. The storage chambers thus can internally communicate with each other through the opening of the one storage chamber and the through hole formed in the other storage chamber. The through hole forming unit is not limited to particular units, and examples thereof include a cutter and a screw. The through hole forming unit may be a piercing unit.

The form C is a method using a capsule. In this case, the reagent storage chamber in the main body is a breakable capsule. The capsule is, for example, then broken. The reagent storage chamber and the collection section storage chamber thus can internally communicate with each other.

The form of the reagent storage chamber as a reaction chamber is described below. Specifically, in this form, a collection section storage chamber of the sample collection tool is a reaction chamber. That is, this form is a form D of moving the collection section to cause the storage chambers to communicate with each other and moving the reaction chamber.

For the one-reagent system using the mixed reagent, the reagent storage chamber serves as, for example, a reaction chamber in which the incubation step and the reaction step are performed. Furthermore, it is preferred that the reagent storage chamber further includes, for example, a detection region, and the detection region is formed of a member capable of detecting a fluorescence polarization degree in the collection section storage chamber from outside. In this form, the collection section placed inside the collection section storage chamber is, for example, placed inside the reagent storage chamber after the preparation step to bring the collection section into contact with the mixed reagent in the reagent storage chamber. The incubation step and the reaction step thus can be performed in parallel. Storing the collection section from the collection section storage chamber to the reagent storage chamber can be, for example, a method in which a partition of separating the collection section storage chamber and the reagent storage chamber is broken by the tip of the collection section to move the collection section from the collection section storage chamber to the reagent storage chamber.

For the two-reagent system separately using the reagent for concentration of bacteria and the fluorescently-labeled binding molecule, the first reagent storage chamber serves as, for example, a reaction chamber in which the incubation step is performed, and the second reagent storage chamber serves as, for example, a reaction chamber in which the reaction step is performed. Furthermore, it is preferred that the second storage chamber further includes a detection region, and the detection region is formed of a member capable of detecting a fluorescence polarization degree in the second reagent storage chamber from outside. In this form, the collection section placed inside the collection section storage chamber is, for example, placed inside the first reagent storage chamber after the preparation step to bring the collection section into contact with the reagent for concentration of bacteria in the first reagent storage chamber. The incubation step thus can be performed. The collection section placed inside the first reagent storage chamber is then placed inside the second reagent storage chamber after the incubation step to introduce the bacteria-concentrated reagent after the incubation in the first reagent storage chamber to the second reagent storage chamber. The reaction step thus can be performed.

For the method of the present invention further including a disinfection step in the form of the one-reagent system or the two-reagent system, the disinfectant storage chamber and the collection section storage chamber internally communicate with each other after the detection step to introduce the disinfectant from the disinfectant storage chamber to the collection section storage chamber. The disinfection step thus can be performed.

The following describes exemplary embodiments of the method of the present invention with reference to the drawings. The present invention, however, is by no means limited thereto. Identical parts in the drawings are denoted by identical reference numerals. In the drawings, the structure and the shape of each component may be shown in a simplified form as appropriate for the sake of convenience in illustration, and each component may be shown schematically with a dimension ratio different from the actual dimension ratio. Being above and being below in each drawing is being above and being below in use of the bacterial detection tool. In the sample collection tool of the bacteria detection tool, the side of the collection section is located below the sample collection tool, and the connection section that is connected to the main body is located above the sample collection tool. Each of the embodiments can be described with reference to the other embodiments.

### Embodiment A1

The present embodiment uses a bacterial detection tool configured so that a collection section storage chamber serves as a reaction chamber in which the incubation step and the reaction step are performed and the collection section storage chamber has a detection region. In the present embodiment, the form of the communication between storage chambers is the above-described form A.

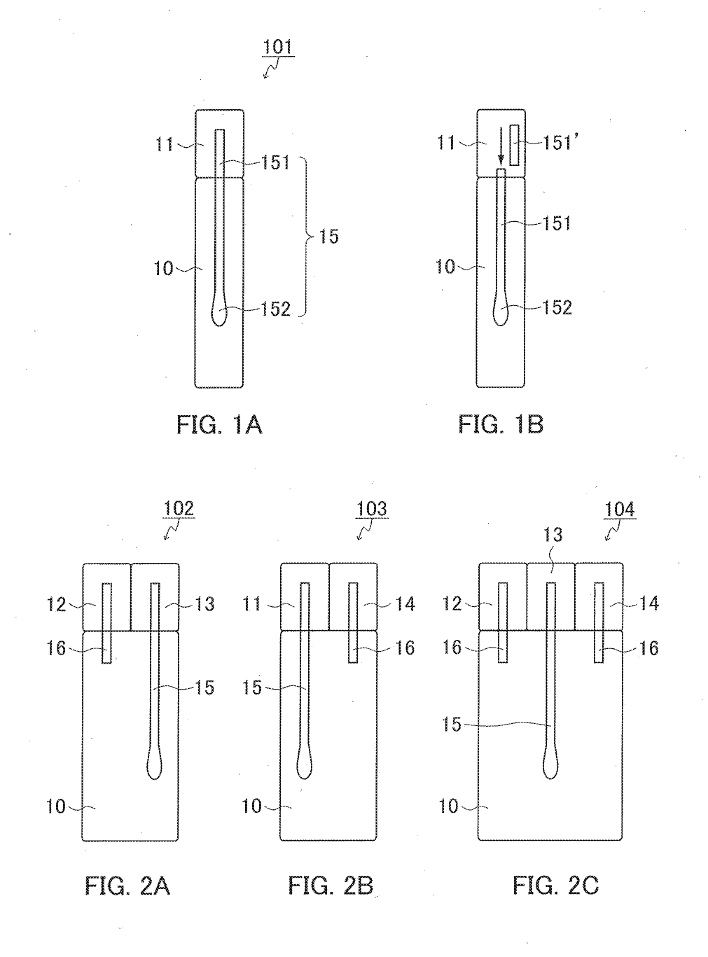

FIGS. 1A and 1B schematically show the bacterial detection tool. FIG. 1A is a sectional view schematically showing the bacterial detection tool. FIG. 1B is a sectional view schematically showing the bacterial detection tool in use.

The bacterial detection tool shown in FIG. 1A includes a collection section storage chamber 10, a reagent storage chamber 11 that contains the above-described mixed reagent, and a sample collection tool 15. In the present embodiment, the main body is constituted by the reagent storage chamber 11 as an upper main body and the collection section storage chamber 10 as a lower main body. The sample collection tool 15 has a hollow tube 151 for communication of the storage chambers and a collection section 152. The collection section 152 is connected to the lower end of the hollow tube 151, and the upper end of the hollow tube 151 has a closed portion. The hollow tube 151 can be cut at any position between both the ends in the axial direction. The hollow tube 151 may have a narrow portion with a smaller diameter at a desired cutting position so as to facilitate cutting, for example. The sample collection tool 15 is connected to the reagent storage chamber 11 as the upper main body. Specifically, the sample collection tool 15 is disposed in such a manner that an upper region of the hollow tube 151 is inside the reagent storage chamber 11. The reagent storage chamber 11 to which the sample collection tool 15 is connected is detachable from the collection section storage chamber 10. The reagent storage chamber 11 can be attached to the collection section storage chamber 10 with the collection section 152 of the sample collection tool 15 being placed inside the collection section storage chamber 10. The collection section storage chamber 10 serves as a reaction chamber in which the incubation step and the reaction step are performed, and a bottom region thereof may be the detection region, for example.

The bacterial detection tool shown in FIG. 1A can be used in the following manner, for example.

First, the reagent storage chamber 11 (upper main body) provided with the sample collection tool 15 is detached from the collection section storage chamber 10 (lower main body) of the bacterial detection tool, and a sample is collected using the thus-exposed tip (the collection section) of the sample collection tool 15. Then, the reagent storage chamber 11 (upper main body) is attached to the collection section storage chamber 10 (lower main body) with the collection section of the sample collection tool 15 being placed inside the collection section storage chamber 10 (lower main body).

Next, as shown in FIG. 1B, the hollow tube 151 of the sample collection tool 15 is cut at a certain position between both the ends in the axial direction, thereby removing a region 151′ having the closed portion from the hollow tube 151. As a result, both the ends of the hollow tube 151 are open, whereby the mixed reagent in the reagent storage chamber 11 is introduced to the hollow tube 151 through the upper opening of the hollow tube 151, and then to the collection section storage chamber (reaction chamber) 10 via the collection section 152 of the sample collection tool 15. At this time, the collected sample adheres to the collection section 152 of the sample collection tool 15. Thus, the mixed reagent that has reached the collection section 152 is mixed with the sample, and the mixture of the mixed reagent and the sample drips onto the bottom of the collection section storage chamber 10.

The mixture of the mixed reagent and the sample introduced to the collection section storage chamber 10 is then subjected to the incubation step and the reaction step, whereby target bacterial cells in the sample are cultured and a binding reaction is caused between the target bacterial cells and the fluorescently-labeled binding molecules. Thereafter, a fluorescence polarization degree is measured in the detection region of the collection section storage chamber 10.

The bacterial detection tool shown in FIGS. 1A and 1B is configured so that a supporting member of the sample collection tool 15 is a hollow tube and the reagent storage chamber 11 and the collection section storage chamber 10 internally communicate with each other. However, the present invention is not limited thereto. For example, the bacterial detection tool may be configured so that the reagent storage chamber 11 and the collection section storage chamber 10 are connected to each other by inserting the hollow tube 151 having a closed portion at the upper end and the sample collection tool 15 is connected only to the bottom of the reagent storage chamber 11.

### Embodiment A2

A bacterial detection tool according to the present embodiment has the same configuration as the bacterial detection tool according to Embodiment A1, except that the number of reagent storage chambers is different or it further includes a disinfectant storage chamber. Unless otherwise stated, the descriptions in Embodiment A1 above and Embodiment A3 to be described below are applicable to the present embodiment.

FIGS. 2A, 2B, and 2C schematically show other examples of the bacterial detection tool. FIG. 2A shows a bacterial detection tool that includes, above a collection section storage chamber 10 (lower main body), an upper main body constituted by a first reagent storage chamber 13 that contains the above-described bacterial concentration reagent and a second reagent storage chamber 12 that contains the above-described fluorescently-labeled binding molecules. FIG. 2B shows a bacterial detection tool that includes, above a collection section storage chamber 10 (lower main body), an upper main body constituted by a reagent storage chamber 11 that contains the above-described mixed reagent and a disinfectant storage chamber 14 that contains the above-described disinfectant. FIG. 2C shows a bacterial detection tool that includes, above a collection section storage chamber 10 (lower main body), an upper main body constituted by a first reagent storage chamber 13 that contains the above-described bacterial concentration reagent, a second reagent storage chamber 12 that contains the above-described fluorescently-labeled binding molecules, and a disinfectant storage chamber 14 that contains the above-described disinfectant.

The first reagent storage chamber 13 and the second reagent storage chamber 12 in FIG. 2A, the reagent storage chamber 11 and the disinfectant storage chamber 14 in FIG. 2B, and the first reagent storage chamber 13, the second reagent storage chamber 12, and the disinfectant storage chamber 14 in FIG. 2C are separated from each other with partition walls or the like. Preferably, the adjacent storage chambers to be paired are connected to each other with the partition wall, so that the respective chambers are integrated as a whole. With this configuration, when the first reagent storage chamber 13, the second reagent storage chamber 12, the reagent storage chamber 11, or the disinfectant storage chamber 14 is detached from the collection section storage chamber 10 (lower main body), the remaining storage chambers also can be detached from the collection section storage chamber 10 together with the detached chamber.

The first reagent storage chamber 13 and the collection section storage chamber 10 in FIGS. 2A and 2C are connected to each other by inserting the hollow tube 151 of the sample collection tool 15. As to the relationship among the first reagent storage chamber 13, the collection section storage chamber 10, and the sample collection tool 15, reference can be made to the above description regarding the relationship among the reagent storage chamber 11, the collection section storage chamber 10, and the sample collection tool 15 in FIG. 1A of Embodiment A1. The relationship among the reagent storage chamber 11, the collection section storage chamber 10, and the sample collection tool 15 in FIG. 2B is the same as that in FIG. 1A of Embodiment A1.

The second reagent storage chamber 12 and the collection section storage chamber 10 in FIGS. 2A and 2C, and the disinfectant storage chamber 14 and the collection section storage chamber 10 in FIG. 2B are connected to each other by inserting a hollow tube 16 having a closed portion at an upper end. The hollow tube 16 has the same configuration as the hollow tube 151 of the sample collection tool 15 in FIGS. 1A and 1B of Embodiment A1, and has a closed portion at the upper end and can be cut at any position between both ends in the axial direction.

The bacterial detection tool shown in FIG. 2A can be used in the following manner, for example. First, the upper main body provided with the sample collection tool 15 is detached from the collection section storage chamber 10 (lower main body) of the bacterial detection tool, and a sample is collected. Thereafter, the upper main body is attached to the lower main body again, so that the collection section of the sample collection tool 15 is placed inside the collection section storage chamber 10 (lower main body). Next, the hollow tube of the sample collection tool 15 is cut to introduce the bacterial concentration reagent from the first reagent storage chamber 13 to the collection section storage chamber 10, and the incubation step is performed. Further, the hollow tube 16 of the second reagent storage chamber 12 is cut to introduce the fluorescently-labeled binding molecules from the second reagent storage chamber 12 to the collection section storage chamber 10, and the reaction step is performed. Then, a fluorescence polarization degree is detected in a detection region of the collection section storage chamber 10.

Next, the bacterial detection tool shown in FIG. 2B can be used in the following manner, for example. First, the upper main body provided with the sample collection tool 15 is detached from the collection section storage chamber 10 (lower main body) of the bacterial detection tool, and a sample is collected. Thereafter, the upper main body is attached to the lower main body again, so that the collection section of the sample collection tool 15 is placed inside the collection section storage chamber 10 (lower main body). Then, the hollow tube of the sample collection tool 15 is cut to introduce the mixed reagent from the reagent storage chamber 11 to the collection section storage chamber 10, and the incubation step and the reaction step are performed. Thereafter, a fluorescence polarization degree is detected in the detection region of the collection section storage chamber 10. Further, the hollow tube 16 of the disinfectant storage chamber 14 is cut to introduce the disinfectant from the disinfectant storage chamber 14 to the collection section storage chamber 10, and the sterilization step is performed.

Next, the bacterial detection tool shown in FIG. 2C can be used in the following manner, for example. First, the upper main body provided with the sample collection tool 15 is detached from the collection section storage chamber 10 (lower main body) of the bacterial detection tool, and a sample is collected. Thereafter, the upper main body is attached to the lower main body again, so that the collection section of the sample collection tool 15 is placed inside the collection section storage chamber 10 (lower main body). Then, the hollow tube of the sample collection tool 15 is cut to introduce the bacterial concentration reagent from the first reagent storage chamber 13 to the collection section storage chamber 10, and the incubation step is performed. Further, the hollow tube 16 of the second reagent storage chamber 12 is cut to introduce the fluorescently-labeled binding molecules from the second reagent storage chamber 12 to the collection section storage chamber 10, and the reaction step is performed. Thereafter, a fluorescence polarization degree is detected in the detection region of the collection section storage chamber 10. Then, the hollow tube 16 of the disinfectant storage chamber 14 is cut to introduce the disinfectant from the disinfectant storage chamber 14 to the collection section storage chamber 10, and the sterilization step is performed.

### Embodiment A3

A bacterial detection tool according to the present embodiment has the same configuration as the bacterial detection tools according to Embodiments A1 and A2, except that a reagent storage chamber is disposed differently from those in Embodiments A1 and A2.

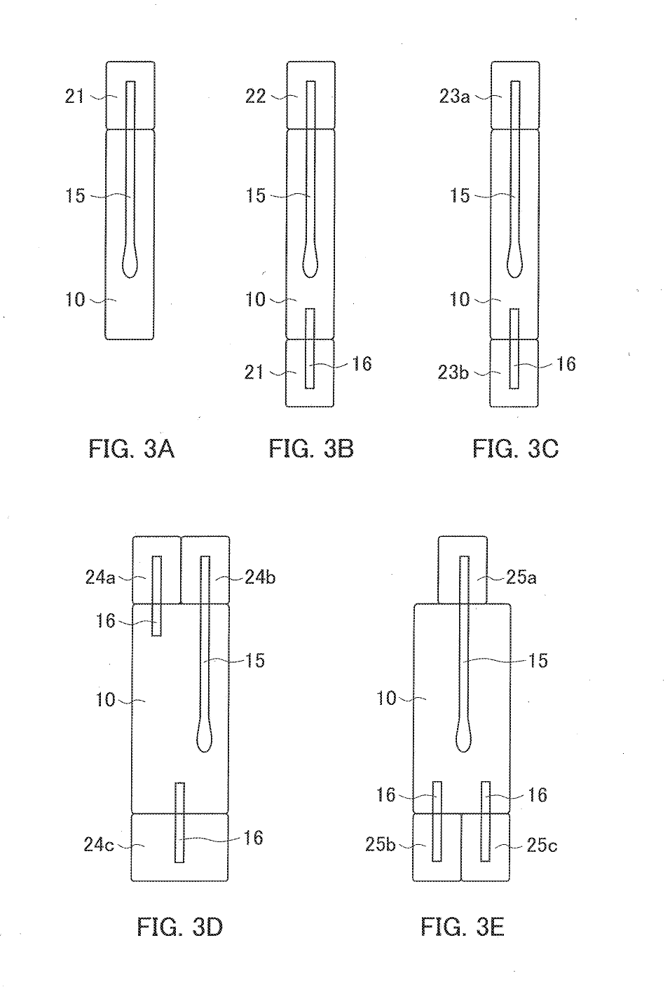

FIGS. 3A, 3B, 3C, 3D, and 3E schematically show still other examples of the bacterial detection tool. A bacterial detection tool shown in FIG. 3A is configured so that, similarly to the bacterial detection tools of Embodiments A1 and A2, a collection section storage chamber 10 constitutes a lower main body, and a storage chamber 21 provided with the sample collection tool 15 constitutes an upper main body. In FIG. 3A, the storage chamber 21 may have the configuration of the upper main body in either Embodiment A1 or A2, for example. That is, the storage chamber 21 may be, for example, the reagent storage chamber 11 in Embodiment A1, the upper main body (the first reagent storage chamber 13 and the second reagent storage chamber 12) shown in FIG. 2A in Embodiment A2, the upper main body (the reagent storage chamber 11 and the disinfectant storage chamber 14) shown in FIG. 2B in Embodiment A2, or the upper main body (the first reagent storage chamber 13, the second reagent storage chamber 12, and the disinfectant storage chamber 14) shown in FIG. 2C in Embodiment A2. The bacterial detection tool shown in FIG. 3A can be used in the same manner as the bacterial detection tools of Embodiments A1 and A2.

FIG. 3B shows a bacterial detection tool configured so that a collection section storage chamber 10 constitutes an upper main body and a storage chamber for a reagent or the like constitutes a lower main body. The bacterial detection tool shown in FIG. 3B includes a sample collection tool 15, a handle section 22 to which the sample collection tool 15 is fixed, a collection section storage chamber 10, and storage chamber 21. The sample collection tool 15 constitutes the upper main body, and the storage chamber 21 constitutes the lower main body. The handle section 22 to which the sample collection tool 15 is fixed can be attached to the collection section storage chamber 10 (upper main body) with the collection section of the sample collection tool 15 being placed inside the collection section storage chamber 10 (upper main body). In the present embodiment, the storage chamber 21 has the same configuration as the upper main bodies in Embodiments A1 and A2, except that the storage chamber 21 is disposed below the collection section storage chamber 10.

In the bacterial detection tool shown in FIG. 3B, first, the handle section 22 provided with the sample collection tool 15 is detached from the collection section storage chamber 10 (upper main body) of the bacterial detection tool, and a sample is collected. Thereafter, the handle section 22 is attached to the upper main body again, so that the collection section of the sample collection tool 15 is placed inside the collection section storage chamber 10 (upper main body). After the attachment, the bacterial detection tool is turned upside down so that the storage chamber 21 is located above the collection section storage chamber 10. The reagent or disinfectant in the storage chamber 21 is then introduced to the collection section storage chamber 10 in the same manner as in Embodiments 1A and 2A.

FIG. 3C shows a bacterial detection tool configured so that two or more storage chambers are disposed above and below the collection section storage chamber 10. The bacterial detection tool shown in FIG. 3C includes a sample collection tool 15, a collection section storage chamber 10, an upper storage chamber 23a, and a lower storage chamber 23b. The upper storage chamber 23a constitutes an upper main body, the collection section storage chamber 10 constitutes a middle main body, and the lower storage chamber 23b constitutes a lower main body. In the present embodiment, the combination of the storage chamber 23a (upper main body) and the storage chamber 23b (lower main body) is not particularly limited, and the mixed reagent storage chamber, the first reagent storage chamber, the second reagent storage chamber, and the disinfectant storage chamber may be combined as appropriate. Specific examples of the combination include the following combinations: the first reagent storage chamber constitutes the upper main body and the second reagent storage chamber constitutes the lower main body; and the mixed reagent storage chamber constitutes the upper main body and the disinfectant storage chamber constitutes the lower main body. The bacterial detection tool shown in FIG. 3C can be used in the same manner as the bacterial detection tools of Embodiments 1A and 2A, except that, after collecting a sample, the upper main body 23a provided with the sample collection tool 15 is attached to the collection section storage chamber 10 (middle main body) connected to the lower main body 23b, and the bacterial detection tool is turned upside down when necessary.

FIG. 3D shows a bacterial detection tool configured so that three or more storage chambers are disposed above and below the collection section storage chamber 10. The bacterial detection tool shown in FIG. 3D includes a sample collection tool 15, a collection section storage chamber 10, upper storage chambers 24a and 24b, and a lower storage chamber 24c. The upper storage chambers 24a and 24b constitute an upper main body, the collection section storage chamber 10 constitutes a middle main body, and the lower storage chamber 24c constitutes a lower main body. In the present embodiment, the combination of the storage chambers 24a and 24b (upper main body) and the storage chamber 24c (lower main body) is not particularly limited, and the mixed reagent storage chamber, the first reagent storage chamber, the second reagent storage chamber, and the disinfectant storage chamber may be combined as appropriate. Specific examples of the combination include the following combination: the first reagent storage chamber 24b and the second reagent storage chamber 24a constitute the upper main body and the disinfectant storage chamber 24c constitutes the lower main body. The bacterial detection tool shown in FIG. 3D can be used in the same manner as the bacterial detection tools of Embodiments 1A and 2A, except that, after collecting a sample, the upper main body (24a and 24b) provided with the sample collection tool 15 is attached to the collection section storage chamber 10 (middle main body) connected to the lower main body 24c, and the bacterial detection tool is turned upside down when necessary.

FIG. 3E shows a bacterial detection tool configured so that three or more storage chambers are disposed above and below the collection section storage chamber 10. The bacterial detection tool shown in FIG. 3E includes a sample collection tool 15, a collection section storage chamber 10, an upper storage chamber 25a, and lower storage chambers 25b and 25c. The upper storage chamber 25a constitutes an upper main body, the collection section storage chamber 10 constitutes a middle main body, and the lower storage chambers 25b and 25c constitute a lower main body. In the present embodiment, the combination of the storage chamber 25a (upper main body) and the storage chambers 25b and 25c (lower main body) is not particularly limited, and the mixed reagent storage chamber, the first reagent storage chamber, the second reagent storage chamber, and the disinfectant storage chamber may be combined as appropriate. Specific examples of the combination include the following combination: the first reagent storage chamber 25a constitutes the upper main body, and the second reagent storage chamber 25b and the disinfectant storage chamber 25c constitute the lower main body. The bacterial detection tool shown in FIG. 3E can be used in the same manner as the bacterial detection tools of Embodiments 1A and 2A, except that, after collecting a sample, the upper main body (25a) provided with the sample collection tool 15 is attached to the collection section storage chamber 10 (middle main body) connected to the lower main body (24b and 24c), and the bacterial detection tool is turned upside down when necessary.

### Embodiment B1

The present embodiment uses a bacterial detection tool configured so that a collection section storage chamber serves as a reaction chamber in which the incubation step and the reaction step are performed and the collection section storage chamber has a detection region. In the present embodiment, the form of the communication between storage chambers is the above-described form B, which uses a through hole forming unit.

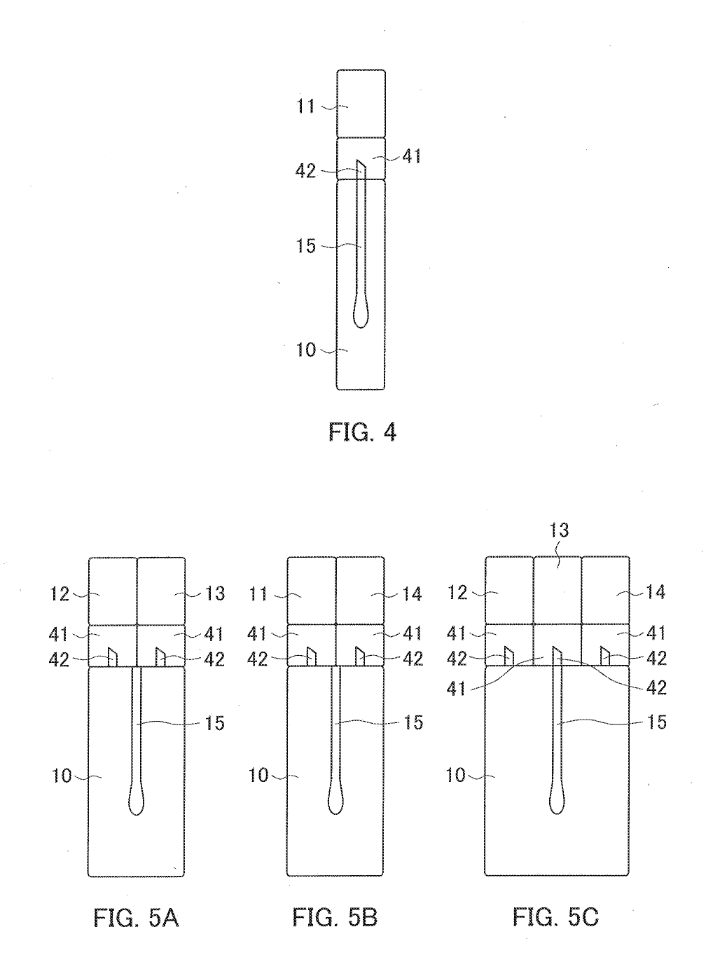

FIG. 4 schematically shows a bacterial detection tool. FIG. 4 is a sectional view schematically showing the bacterial detection tool. The bacterial detection tool shown in FIG. 4 includes a collection section storage chamber 10, a reagent storage chamber 11 that contains the above-described mixed reagent, a sample collection tool 15, and a piercing chamber 41 having a cutter 42 fixed thereto as the through hole forming unit. In the present embodiment, the reagent storage chamber 11 constitutes an upper main body, and the collection section storage chamber 10 constitutes a lower main body. The reagent storage chamber 11 (upper main body) is disposed above the piercing chamber 41. By moving the reagent storage chamber 11 toward the piercing chamber 41 so as to be in contact with the cutter 42 provided in the piercing chamber 41, a through hole can be formed in the bottom of the reagent storage chamber 11. The piercing chamber 41 has a through hole communicating with the collection section storage chamber 10 (lower main body). On an outer surface of the bottom of the piercing chamber 41, the sample collection tool 15 is fixed at a position where the sample collection tool 15 does not overlap the through hole. The piercing chamber 41 to which the reagent storage chamber 11 and the sample collection tool 15 are connected is detachable from the collection section storage chamber 10. The piercing chamber 41 can be attached to the collection section storage chamber 10 with the collection section of the sample collection tool 15 being placed inside the collection section storage chamber 10. The collection section storage chamber 10 serves as a reaction chamber in which the incubation step and the reaction step are performed, and a bottom region thereof may be the detection region, for example.

Since a through hole is formed in the bottom of the reagent storage chamber 11 with the cutter 42, the bottom of the reagent storage chamber 11 preferably is formed of a breakable member, for example. Examples of the member include sheets such as an aluminum sheet.

The bacterial detection tool shown in FIG. 4 can be used in the following manner, for example. First, the piercing chamber 41 provided with the reagent storage chamber 11 (upper main body) and the sample collection tool 15 is detached from the collection section storage chamber 10 (lower main body) of the bacterial detection tool, and a sample is collected. Thereafter, the piercing chamber 41 is attached to the lower main body again, so that the collection section of the sample collection tool 15 is placed inside the collection section storage chamber (lower main body). Next, the sample storage chamber 11 is pushed inside the piercing chamber 41, whereby a through hole is formed in the bottom of the sample storage chamber 11 with the cutter 42 of the piercing chamber 41. As a result of the formation of the through hole, the mixed reagent in the sample storage chamber 11 is introduced to the piercing chamber 41, and the mixed reagent is then introduced to the collection section storage chamber (reaction chamber) 10 through the through hole of the piercing chamber 41. This allows the mixed reagent introduced to the collection section storage chamber 10 to be in contact with the sample on the collection section of the sample collection tool 15. Thereafter, the mixture of the mixed reagent and the sample is subjected to the incubation step and the reaction step. Then, a fluorescence polarization degree is detected in the detection region of the collection section storage chamber 10.

### Embodiment B2

A bacterial detection tool according to the present embodiment has the same configuration as the bacterial detection tool according to Embodiment B1, except that the number of reagent storage chambers is different or it further includes a disinfectant storage chamber. Unless otherwise stated, the descriptions in Embodiment B1 are applicable to the present embodiment.

FIGS. 5A, 5B, and 5C schematically show still other examples of the bacterial detection tool. FIG. 5A shows a bacterial detection tool that includes, above a collection section storage chamber 10 (lower main body), an upper main body constituted by a first reagent storage chamber 13 that contains the above-described bacterial concentration reagent and a second reagent storage chamber 12 that contains the above-described fluorescently-labeled binding molecules, and further includes piercing chambers 41 provided for the respective storage chambers. FIG. 5B shows a bacterial detection tool that includes, above a collection section storage chamber 10 (lower main body), an upper main body constituted by a reagent storage chamber 11 that contains the above-described mixed reagent and a disinfectant storage chamber 14 that contains the above-described disinfectant, and further includes piercing chambers 41 provided for the respective storage chambers. FIG. 5C shows a bacterial detection tool that includes, above a collection section storage chamber 10 (lower main body), an upper main body constituted by a first reagent storage chamber 13 that contains the above-described bacterial concentration reagent, a second reagent storage chamber 12 that contains the above-described fluorescently-labeled binding molecules, and a disinfectant storage chamber 14 that contains the above-described disinfectant, and further includes piercing chambers 41 provided for the respective storage chambers. Each of the piercing chambers 41 has the same configuration as that in Embodiment B1, and it has a cutter 42 fixed thereto as a through hole forming unit and also has a through hole communicating with the collection section storage chamber 10.

As to the respective storage chambers constituting the upper main body in each of the bacterial detection tools shown in FIGS. 5A, 5B, and 5C, reference can be made to the above descriptions regarding the combination in FIGS. 2A, 2B, and 2C in Embodiment A2.

The bacterial detection tool shown in FIG. 5A can be used in the following manner, for example. First, the piercing chambers 41 provided with the sample collection tool 15 and the storage chambers 12 and 13 are detached from the collection section storage chamber 10 (lower main body) of the bacterial detection tool, and a sample is collected. Thereafter, the piercing chambers 41 are attached to the lower main body again, so that the collection section of the sample collection tool 15 is placed inside the collection section storage chamber 10 (lower main body). Next, the first reagent storage chamber 13 is pushed inside the piercing chamber 41, whereby a through hole is formed in the bottom of the first reagent storage chamber 13 with the cutter 42 of the piercing chamber 41. Thus, the bacterial concentration reagent is introduced from the first reagent storage chamber 13 to the piercing chamber 41, and the bacterial concentration reagent is then introduced to the collection section storage chamber (reaction chamber) 10 through the through hole of the piercing chamber 41. This allows the bacterial concentration reagent introduced to the collection section storage chamber 10 to be in contact with the sample on the collection section of the sample collection tool 15. Thereafter, the mixture of the bacterial concentration reagent and the sample is subjected to the incubation step. Then, the fluorescently-labeled binding molecules are introduced from the second reagent storage chamber 12 to the collection section storage chamber 10 in the same manner as in the above, and the reaction step is performed. Subsequently, a fluorescence polarization degree is detected in a detection region of the collection section storage chamber 10.

The bacterial detection tool shown in FIG. 5B can be used in the following manner, for example. First, the piercing chambers 41 provided with the sample collection tool 15 and the storage chambers 11 and 14 are detached from the collection section storage chamber 10 (lower main body) of the bacterial detection tool, and a sample is collected. Thereafter, the piercing chambers 41 are attached to the lower main body again, so that the collection section of the sample collection tool 15 is placed inside the collection section storage chamber 10 (lower main body). Next, in the same manner as in the above, the mixed reagent is introduced from the reagent storage chamber 11 to the collection section storage chamber 10, the incubation step and the reaction step are performed, and a fluorescence polarization degree is detected in a detection region of the collection section storage chamber 10. Thereafter, the disinfectant is introduced from the disinfectant storage chamber 14 to the collection section storage chamber 10 in the same manner as in the above, and the sterilization step is performed.

The bacterial detection tool shown in FIG. 5C can be used in the following manner, for example. First, the piercing chambers 41 provided with the sample collection tool 15 and the storage chambers 12, 13, and 14 are detached from the collection section storage chamber 10 (lower main body) of the bacterial detection tool, and a sample is collected. Thereafter, the piercing chambers 41 are attached to the lower main body again, so that the collection section of the sample collection tool 15 is placed inside the collection section storage chamber 10 (lower main body). Next, in the same manner as in the above, the bacterial concentration reagent is introduced from the first reagent storage chamber 13 to the collection section storage chamber 10, the incubation step is performed, the fluorescently-labeled binding molecules are then introduced from the second reagent storage chamber 12 to the collection section storage chamber 10, the reaction step is performed, and a fluorescence polarization degree is detected in a detection region of the collection section storage chamber 10. Thereafter, the disinfectant is introduced from the disinfectant storage chamber 14 to the collection section storage chamber 10 in the same manner as in the above, and the sterilization step is performed.

### Embodiment C1

The present embodiment uses a bacterial detection tool configured so that a collection section storage chamber serves as a reaction chamber in which the incubation step and the reaction step are performed and the collection section storage chamber has a detection region. In the present embodiment, the form of the communication between storage chambers is the above-described form C, which uses a capsule.

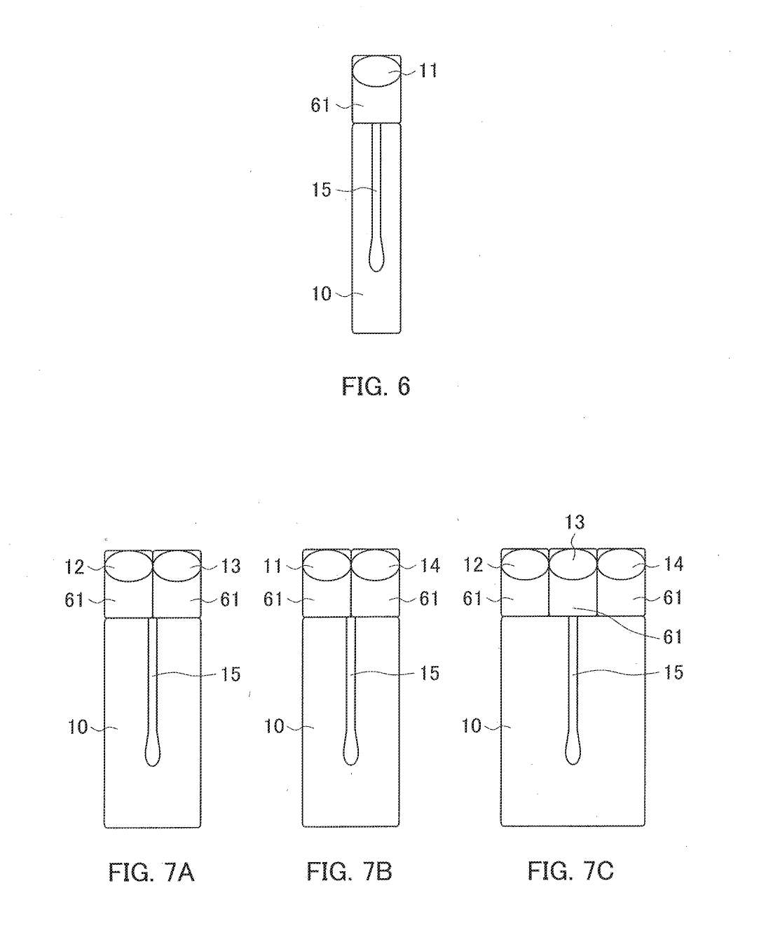

FIG. 6 schematically shows a bacterial detection tool. FIG. 6 is a sectional view showing schematically showing the bacterial detection tool. The bacterial detection tool shown in FIG. 6 includes a sample collection tool 15, a collection section storage chamber 10, a capsule-shaped reagent storage chamber 11 that contains the above-described mixed reagent, and a holding chamber 61 that holds the reagent storage chamber 11. In the present embodiment, the reagent storage chamber 11 and the holding chamber 61 that holds the reagent storage chamber 11 constitute an upper main body, and the collection section storage chamber 10 constitutes a lower main body. The holding chamber 61 has a through hole communicating with the collection section storage chamber 10 (lower main body). On an outer surface of the bottom of the holding chamber 61, the sample collection tool 15 is fixed at a position where the sample collection tool 15 does not overlap the through hole. The holding chamber 61 to which the sample collection tool 15 is connected is detachable from the collection section storage chamber 10. The holding chamber 61 can be attached to the collection section storage chamber 10 with the collection section of the sample collection tool 15 being placed inside the collection section storage chamber 10. The collection section storage chamber 10 serves as a reaction chamber in which the incubation step and the reaction step are performed, and a bottom region thereof may be the detection region, for example.

The bacterial detection tool shown in FIG. 6 can be used in the following manner, for example. First, the holding chamber 61 (upper main body) provided with the capsule-shaped reagent storage chamber 11 and the sample collection tool 15 is detached from the collection section storage chamber 10 (lower main body) of the bacterial detection tool, and a sample is collected. Thereafter, the holding chamber 61 is attached to the lower main body again, so that the collection section of the sample collection tool 15 is placed inside the collection section storage chamber (lower main body). Next, the capsule-shaped reagent storage chamber 11 held inside the holding chamber 61 is ruptured. As a result of the rupture, the mixed reagent in the sample storage chamber 11 is introduced to the holding chamber 61, and the mixed reagent is then introduced to the collection section storage chamber (reaction chamber) 10 through the through hole of the holding chamber 61. This allows the mixed reagent introduced to the collection section storage chamber 10 to be in contact with the sample on the collection section of the sample collection tool 15. Thereafter, the mixture of the mixed reagent and the sample is subjected to the incubation step and the reaction step. Then, a fluorescence polarization degree is detected in a detection region of the collection section storage chamber 10.

### Embodiment C2

A bacterial detection tool according to the present embodiment has the same configuration as the bacterial detection tool according to Embodiment C1, except that the number of reagent storage chambers is different or it further includes a disinfectant storage chamber. Unless otherwise stated, the descriptions in Embodiment C1 are applicable to the present embodiment.

FIGS. 7A, 7B, and 7C schematically show still other examples of the bacterial detection tool. FIG. 7A shows a bacterial detection tool that includes, above a collection section storage chamber 10 (lower main body), an upper main body constituted by a capsule-shaped first reagent storage chamber 13 that contains the above-described bacterial concentration reagent, a holding chamber 61 that holds the capsule-shaped first reagent storage chamber 13, a capsule-shaped second reagent storage chamber 12 that contains the above-described fluorescently-labeled binding molecules, and a holding chamber 61 that holds the capsule-shaped second reagent storage chamber 12. FIG. 7B shows a bacterial detection tool that includes, above a collection section storage chamber 10 (lower main body), an upper main body constituted by a capsule-shaped reagent storage chamber 11 that contains the above-described mixed reagent, a holding chamber 61 that holds the capsule-shaped reagent storage chamber 11, a capsule-shaped disinfectant storage chamber 14 that contains the above-described disinfectant, and a holding chamber 61 that holds the capsule-shaped disinfectant storage chamber 14. FIG. 7C shows a bacterial detection tool that includes, above a collection section storage chamber 10 (lower main body), an upper main body constituted by a capsule-shaped first reagent storage chamber 13 that contains the above-described bacterial concentration reagent, a holding chamber that holds the capsule-shaped first reagent storage chamber 13, a capsule-shaped second reagent storage chamber 12 that contains the above-described fluorescently-labeled binding molecules, a holding chamber that holds the capsule-shaped second reagent storage chamber 12, and a capsule-shaped disinfectant storage chamber 14 that contains the above-described disinfectant, and a holding chamber that holds the capsule-shaped disinfectant storage chamber 14. Each of the holding chambers 61 has the same configuration as that in Embodiment C1, and has a through hole communicating with the collection section storage chamber 10.

As to the respective storage chambers constituting the upper main body in each of the bacterial detection tools shown in FIGS. 7A, 7B, and 7C, reference can be made to the above descriptions regarding the combination in FIGS. 2A, 2B, and 2C in Embodiment A2.

The bacterial detection tool shown in FIG. 7A can be used in the following manner, for example. First, the holding chambers 61 provided with the sample collection tool 15 and the capsule-shaped storage chambers 12 and 13 are detached from the collection section storage chamber 10 (lower main body) of the bacterial detection tool, and a sample is collected. Thereafter, the holding chamber 61s are attached to the lower main body again, so that the collection section of the sample collection tool 15 is placed inside the collection section storage chamber 10 (lower main body). Next, the capsule-shaped first reagent storage chamber 13 in the holding chamber 61 is ruptured. Thus, the bacterial concentration reagent is introduced from the first reagent storage chamber 13 to the holding chamber 61, and the bacterial concentration reagent is introduced to the collection section storage chamber (reaction chamber) 10 through the through hole of the holding chamber 61. This allows the bacterial concentration reagent introduced to the collection section storage chamber 10 to be in contact with the sample on the collection section of the sample collection tool 15. Thereafter, the mixture of the bacterial concentration reagent and the sample is subjected to the incubation step. Then, the fluorescently-labeled binding molecules are introduced from the capsule-shaped second reagent storage chamber 12 to the collection section storage chamber 10 in the same manner as in the above, and the reaction step is performed. Subsequently, a fluorescence polarization degree is detected in a detection region of the collection section storage chamber 10.

The bacterial detection tool shown in FIG. 7B can be used in the following manner, for example. First, the holding chambers 61 provided with the sample collection tool 15 and the capsule-shaped storage chambers 11 and 14 are detached from the collection section storage chamber 10 (lower main body) of the bacterial detection tool, and a sample is collected. Thereafter, the holding chambers 61 are attached to the lower main body again, so that the collection section of the sample collection tool 15 is placed inside the collection section storage chamber 10 (lower main body). Next, in the same manner as in the above, the mixed reagent is introduced from the reagent storage chamber 11 to the collection section storage chamber 10, and the incubation step and the reaction step are performed. Then, a fluorescence polarization degree is detected in a detection region of the collection section storage chamber 10. Thereafter, the disinfectant is introduced to the collection section storage chamber 10 from the capsule-shaped disinfectant storage chamber 14 in the same manner as in the above, and the sterilization step is performed.

The bacterial detection tool shown in FIG. 7C can be used in the following manner, for example. First, the holding chambers 61 provided with the sample collection tool 15 and the capsule-shaped storage chambers 12, 13, and 14 are detached from the collection section storage chamber 10 (lower main body) of the bacterial detection tool, and a sample is collected. Thereafter, the holding chambers 61 are attached to the lower main body again, so that the collection section of the sample collection tool 15 is placed inside the collection section storage chamber 10 (lower main body). Next, the bacterial concentration reagent is introduced to the collection section storage chamber 10 from the capsule-shaped first reagent storage chamber 13, and the incubation step is performed. Then, the fluorescently-labeled binding molecules are introduced from the capsule-shaped second reagent storage chamber 12 to the collection section storage chamber 10 in the same manner as in the above, and the reaction step is performed. Subsequently, a fluorescence polarization degree is detected in a detection region of the collection section storage chamber 10. Thereafter, the disinfectant is introduced to the collection section storage chamber 10 from the capsule-shaped disinfectant storage chamber 14 in the same manner as in the above, and the sterilization step is performed.

### Embodiment D1

The present embodiment uses a bacterial detection tool configured so that each storage chamber serves as a reaction chamber in which the incubation step and the reaction step are performed. In the present embodiment, the form of the communication between the storage chambers is the above-described form D, namely, the communication is achieved utilizing the movement of a collection section of the sample collection tool.

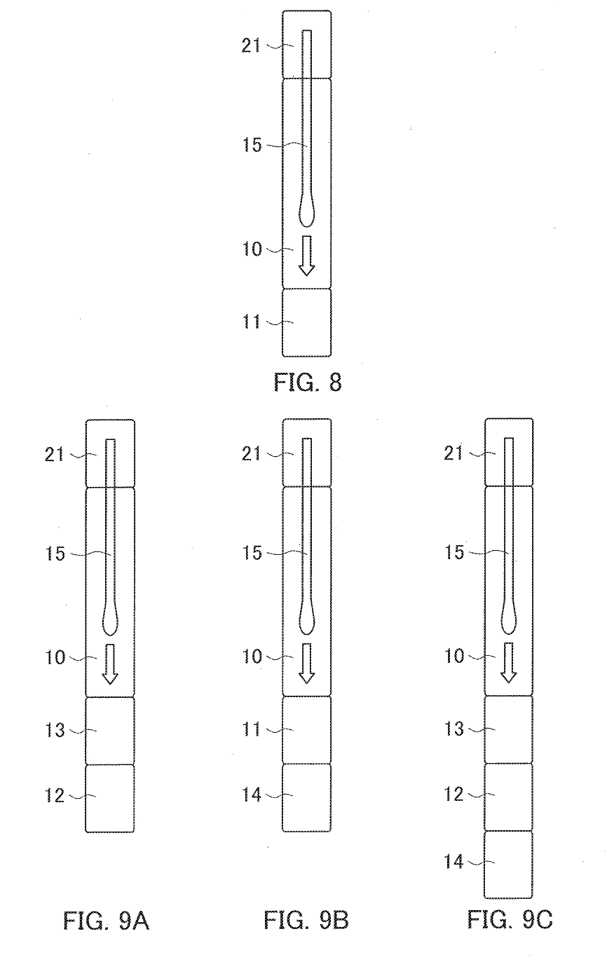

FIG. 8 schematically shows a bacterial detection tool. FIG. 8 is a sectional view schematically showing the bacterial detection tool. The bacterial detection tool shown in FIG. 8 includes a sample collection tool 15, a handle section 21 to which the sample collection tool 15 is fixed, a collection section storage chamber 10, and a reagent storage chamber 11 that contains the above-described mixed reagent. In the present embodiment, the reagent storage chamber 11 is connected to a lower part of the collection section storage chamber 10, and a through hole can be formed in an upper surface of the reagent storage chamber 11 by bringing the collection section of the sample collection tool 15 into contact with the upper surface. The handle section 21 to which the sample collection tool 15 is fixed is detachable from the collection section storage chamber 10. In the present embodiment, the reagent storage chamber 11 serves as a reaction chamber in which the incubation step and the reaction step are performed, and a bottom region thereof may be the above-described detection region, for example.

The bacterial detection tool shown in FIG. 8 can be used in the following manner, for example. First, the handle section 21 to which the sample collection tool 15 is fixed is detached from the collection section storage chamber 10 (upper main body) of the bacterial detection tool, and a sample is collected. Thereafter, the handle section 21 is attached to the lower main body again, so that the collection section of the sample collection tool 15 is placed inside the collection section storage chamber (upper main body). Next, the tip of the sample collection tool 15 in the sample storage chamber 11 is pushed downward to break through the upper surface of the sample storage chamber 11, thereby allowing the collection section of the sample collection tool 15 to be in contact with the mixed reagent in the sample storage chamber 11. As a result, the mixed reagent is mixed with the sample, and the resultant mixture is subjected to the incubation step and the reaction step. Then, a fluorescence polarization degree is detected in the detection region in the sample storage chamber 11.

### Embodiment D2

A bacterial detection tool according to the present embodiment has the same configuration as the bacterial detection tool according to Embodiment D1, except that the number of reagent storage chambers is different or it further includes a disinfectant storage chamber. Unless otherwise stated, the descriptions in Embodiment D1 are applicable to the present embodiment.

FIGS. 9A, 9B, and 9C schematically show still other examples of the bacterial detection tool. FIG. 9A shows a bacterial detection tool that includes, below a collection section storage chamber 10, a first reagent storage chamber 13 that contains the above-described bacterial concentration reagent and a second reagent storage chamber 12 that contains the above-described fluorescently-labeled binding molecules in this order. FIG. 9B shows a bacterial detection tool that includes, below a collection section storage chamber 10, a reagent storage chamber 11 that contains the above-described mixed reagent and a disinfectant storage chamber 14 that contains the above-described disinfectant in this order. FIG. 9C shows a bacterial detection tool that includes, below a collection section storage chamber 10, a first reagent storage chamber 13 that contains the above-described bacterial concentration reagent, a second reagent storage chamber 12 that contains the above-described fluorescently-labeled binding molecules, and a disinfectant storage chamber 14 that contains the above-described disinfectant in this order. In each of FIGS. 9A, 9B, and 9C, a sample collection tool 15 has the same configuration as that in Embodiment D1, and is fixed to a handle section 21. The handle section 21 is detachable from the collection section storage chamber 10.

The bacterial detection tool shown in FIG. 9A can be used in the following manner, for example. First, the handle section 21 provided with the sample collection tool 15 is detached from the collection section storage chamber 10 (upper main body) of the bacterial detection tool, and a sample is collected. Thereafter, the handle section 21 is attached to the upper main body again, so that the collection section of the sample collection tool 15 is placed inside the collection section storage chamber 10 (upper main body). Next, the tip of the sample collection tool 15 in the collection section storage chamber 10 is pushed downward to break through an upper surface of the first reagent storage chamber 13, thereby allowing the collection section of the sample collection tool 15 to be in contact with the bacterial concentration reagent in the first reagent storage chamber 13. As a result, the bacterial concentration reagent is mixed with the sample, and the resultant mixture is subjected to the incubation step. Next, the tip of the sample collection tool 15 in the first reagent storage chamber 13 is further pushed downward to break through an upper surface of the second reagent storage chamber 12. Thus, a through hole is formed in the upper surface, and the mixture in the first reagent storage chamber 13 is introduced to the second reagent storage chamber 12. As a result, the mixture having been subjected to the incubation step is mixed with the fluorescently-labeled binding molecules, and the resultant mixture is subjected to the reaction step. Then, a fluorescence polarization degree is detected in a detection region of the second reagent storage chamber 12.

The bacterial detection tool shown in FIG. 9B can be used in the following manner, for example. First, the handle section 21 provided with the sample collection tool 15 is detached from the collection section storage chamber 10 (upper main body) of the bacterial detection tool, and a sample is collected. Thereafter, the handle section 21 is attached to the upper main body again, so that the collection section of the sample collection tool 15 is placed inside the collection section storage chamber 10 (upper main body). Next, the tip of the sample collection tool 15 in the collection section storage chamber 10 is pushed downward to break through an upper surface of the sample storage chamber 11, thereby allowing the mixed reagent in the sample storage chamber 11 to be in contact with the collection section of the sample collection tool 15. As a result, the mixed reagent is mixed with the sample, and the resultant mixture is subjected to the incubation step and the reaction step. Thereafter, a fluorescence polarization degree is detected in a detection region of the sample storage chamber 11. Next, the tip of the sample collection tool 15 in the reagent storage chamber 11 is further pushed downward to break through an upper surface of the disinfectant storage chamber 14. Thus, a through hole is formed in the upper surface, and the mixture in the reagent storage chamber 11 is introduced to the disinfectant storage chamber 14. As a result, the mixture having been subjected to the detection is mixed with the disinfectant, and the sterilization step is performed.

The bacterial detection tool shown in FIG. 9C can be used in the following manner, for example. First, the handle section 21 provided with the sample collection tool 15 is detached from the collection section storage chamber 10 (upper main body) of the bacterial detection tool, and a sample is collected. Thereafter, the handle section 21 is attached to the upper main body again, so that the collection section of the sample collection tool 15 is placed inside the collection section storage chamber 10 (upper main body). Next, the tip of the sample collection tool 15 in the collection section storage chamber 10 is pushed downward to break through an upper surface of the first reagent storage chamber 13, thereby allowing the bacterial concentration reagent in the first reagent storage chamber 13 to be in contact with the collection section of the sample collection tool 15. As a result, the bacterial concentration reagent is mixed with the sample, and the resultant mixture is subjected to the incubation step. Next, the tip of the sample collection tool 15 in the first reagent storage chamber 13 is further pushed downward to break through an upper surface of the second reagent storage chamber 12. Thus, a through hole is formed in the upper surface, and the mixture in the first reagent storage chamber 13 is introduced to the second reagent storage chamber 12. As a result, the mixture having been subjected to the incubation step is mixed with the fluorescently-labeled binding molecules, and the resultant mixture is subjected to the reaction step. Then, a fluorescence polarization degree is detected in a detection region of the second reagent storage chamber 12. The tip of the sample collection tool 15 in the second reagent storage chamber 12 is further pushed downward to break through an upper surface of the disinfectant storage chamber 14. Thus, a through hole is formed in the upper surface, and the mixture in the second reagent storage chamber 11 is introduced to the disinfectant storage chamber 14. As a result, the mixture having been subjected to the detection is mixed with the disinfectant, and the sterilization step is performed.

For example, the size and the shape of the bacterial detection tool used in the method of the present invention is not limited to particular sizes and shapes. In the bacterial detection tool, the shape of each storage chamber is, for example, a tubular shape, and specific examples thereof include a cylindrical shape and a rectangular shape. Examples of the cylindrical shape include an exact circle and an ellipse. Examples of the rectangular shape include: boxes such as a square and a rectangle; and polygons.

The linkage and the attachment of each component in the bacterial detection tool are not limited to particular linkages and attachments, and examples thereof include screwing, fitting, and inserting. The inserting is, for example, preferably forcibly inserting. For the linkage of each component, a combination of a male die and a female dime can be used, and examples of the combination include a combination of a projection and a depression that corresponds to each other, and a combination of a male screw and a female screw.

[2. Bacteria Detection Tool]

As mentioned above, the bacterial detection tool of the present invention, for use in the method of the present invention includes:

a sample collection tool; and

a main body, wherein

the sample collection tool includes:
a collection section in which a sample is collected; and
a connection section that is connected to the main body,

the main body includes:
a reagent storage chamber that contains the reagent and the fluorescently-labeled binding molecule;
a collection section storage chamber that contains the collection section of the sample collection tool; and
a connection section that is connected to the sample collection tool,

the reagent storage chamber and the collection section storage chamber are separated from each other, and

the sample collection tool and the main body are connected to each other at the connection section of the sample collection tool and the connection section of the main body with the collection section of the sample collection tool being placed inside the collection section storage chamber of the main body.

The bacterial detection tool of the present invention is for use in the method of the present invention and can be described with reference to the whole description of the bacterial detection tool in the method of the present invention.

The present invention is described above with reference to the exemplary embodiments. The present invention, however, is by no means limited thereto. Various changes and modifications that may become apparent to those skilled in the art may be made in the configuration and specifics of the present invention without departing from the scope of the present invention.

This application is based upon and claims the benefit of priority from Japanese patent application No. 2014-225682, filed on Nov. 6, 2014, the disclosure of which is incorporated herein its entirety by reference.

### INDUSTRIAL APPLICABILITY

The present invention can simply and easily cultivate bacteria in a sample and detect target bacteria. Specifically, the present invention can detect target bacteria from the change in fluorescence polarization degree using a fluorescently-labeled aptamer as a reagent. This enables a simple detection. The present invention therefore is really useful in the fields of, for example, food manufacturing, food management, and food distribution.

### EXPLANATION OF REFERENCE NUMERALS

10 collection section storage chamber
11 reagent storage chamber
12 second reagent storage chamber
13 first reagent storage chamber
14 disinfectant storage chamber
15 sample collection tool
16 hollow tube
21 handle section
41 piercing chamber
42 cutter
61 holding chamber

1. A method for detecting a bacterial cell comprising:
a preparation step of preparing a sample;
an incubation step of incubating the sample in a reagent for concentration of bacteria;
a reaction step of causing the sample in the reagent and a fluorescently-labeled binding molecule to be bonded to a target bacterium to react with each other; and
a detection step of detecting a fluorescence polarization degree of the fluorescently-labeled binding molecule to detect the target bacterium, wherein
the preparation step is performed using a sample collection tool,
the incubation step, the reaction step, and the detection step are performed using a bacterial detection tool obtained by attaching the sample collection tool to a main body,
the sample collection tool comprises:
a collection section in which the sample is collected; and
a connection section that is connected to the main body,
the main body comprises:
a reagent storage chamber that contains the reagent and the fluorescently-labeled binding molecule;
a collection section storage chamber that contains the collection section of the sample collection tool; and
a connection section that is connected to the sample collection tool,
the reagent storage chamber and the collection section storage chamber are separated from each other,
the sample collection tool and the main body are connected to each other at the connection section of the sample collection tool and the connection section of the main body after the preparation step and before the incubation step with the collection section of the sample collection tool being placed inside the collection section storage chamber of the main body, and
the reagent storage chamber and the collection section storage chamber internally communicate with each other after the preparation step to perform the incubation step and the reaction step.

2. The method according to claim 1, wherein
the reagent storage chamber contains a mixed reagent of the reagent and the fluorescently-labeled binding molecule, and
the reagent storage chamber and the collection section storage chamber internally communicate with each other after the preparation step to bring the sample in the collection section of the reagent collection tool and the mixed reagent into contact with each other and perform the incubation step and the reaction step in parallel.

3. The method according to claim 1, wherein
the reagent storage chamber of the main body in use of the bacterial detection tool is disposed above or below the collection section storage chamber.

4. The method according to claim 1, wherein
the reagent storage chamber separately comprises:
a first reagent storage chamber; and
a second reagent storage chamber,
the first reagent storage chamber contains the reagent,
the second reagent storage chamber contains the fluorescently-labeled binding molecule,
the first reagent storage chamber and the second reagent storage chamber are separated from the collection section storage chamber,
the first reagent storage chamber and the collection section storage chamber internally communicate with each other after the preparation step to bring the sample in the collection section of the sample collection tool and the reagent into contact with each other and perform the incubation step, and
the second reagent storage chamber and the collection section storage chamber internally communicate with each other after the incubation step to bring the incubated sample and the fluorescently-labeled binding molecule into contact with each other and perform the reaction step.

5. The method according to claim 4, wherein
the first reagent storage chamber and the second reagent storage chamber in the main body in use of the bacterial detection tool are both disposed above or below the collection section storage chamber.

6. The method according to claim 4, wherein
either one of the first reagent storage chamber and the second reagent storage chamber in the main body in use of the bacterial detection tool is disposed above the collection section storage chamber, and the other is disposed below the collection section storage chamber.

7. The method according to any one of claim 1, further comprising:
a disinfection step of treating the incubated sample with a disinfectant after the detection step, wherein
the main body further comprises:
a disinfectant storage chamber that contains the disinfectant,
the reagent storage chamber and the collection section storage chamber are separated from the disinfectant storage chamber, and
the disinfectant storage chamber and the collection section storage chamber internally communicate with each other after the detection step to perform the disinfection step.

8. The method according to claim 7, wherein
the disinfectant storage chamber in the main body in use of the bacterial detection tool is disposed above or below the collection section storage chamber.

9. The method according to claim 1, wherein
the collection section storage chamber serves as a reaction chamber in which the incubation step and the reaction step are performed and further comprises a detection region,
the detection region is formed of a member capable of detecting a fluorescence polarization degree in the collection section storage chamber from outside,
the reagent storage chamber contains a mixed reagent of the reagent and the fluorescently-labeled binding molecule, and
the reagent storage chamber and the collection section storage chamber internally communicate with each other after the preparation step to introduce the mixed reagent from the reagent storage chamber to the collection section storage chamber and perform the incubation step and the reaction step in parallel.

10. The method according to claim 1, wherein
the collection section storage chamber serves as a reaction chamber in which the incubation step and the reaction step are performed and further comprises a detection region,
the detection region is formed of a member capable of detecting a fluorescence polarization degree in the collection section storage chamber from outside,
the reagent storage chamber separately comprises:
a first reagent storage chamber; and
a second reagent storage chamber,
the first reagent storage chamber contains the reagent,
the second reagent storage chamber contains the fluorescently-labeled binding molecule,
the first reagent storage chamber and the second reagent storage chamber are separated from the collection section storage chamber,
the first reagent storage chamber and the collection section storage chamber internally communicate with each other after the preparation step to introduce the reagent from the first reagent storage chamber to the collection section storage chamber and perform the incubation step, and
the second reagent storage chamber and the collection section storage chamber internally communicate with each other after the incubation step to introduce the fluorescently-labeled binding molecule from the second reagent storage chamber to the collection section storage chamber and perform the reaction step.

11. The method according to claim 1, further including:
a disinfection step of treating the incubated sample with a disinfectant after the detection step, wherein
the main body further comprises a disinfectant storage chamber that contains the disinfectant,
the reagent storage chamber and the collection section storage chamber are separated from the disinfectant storage chamber, and
the disinfectant storage chamber and the collection section storage chamber internally communicate with each other after the detection step to introduce the disinfectant from the disinfectant storage chamber to the collection section storage chamber and perform the disinfection step.

12. The method according to claim 9, wherein
the storage chambers to be paired and communicate with each other in the main body are connected to each other by inserting a hollow tube for communication,
the hollow tube comprises a closed portion at one end,
the storage chambers do not internally communicate with each other by the closed portion of the hollow tube, and
the hollow tube is cut at a certain position between both ends in the axial direction to open the both ends of the hollow tube and cause the storage chambers to internally communicate with each other.

13. The method according to claim 9, wherein
the main body further comprises a through hole forming unit,
either one of the storage chambers to be paired and communicate with each other in the main body comprises an opening capable of communicating with the other storage chamber, and the other storage chamber is capable of forming an opening by the through hole forming unit, and
a trough hole is formed in the other chamber by the through hole forming unit to cause the storage chambers to internally communicate with each other through the opening in the one storage chamber and the through hole formed in the other storage chamber.

14. The method according to claim 9, wherein
the reagent storage chamber in the main body is a breakable capsule, and
the capsule is broken to cause the reagent storage chamber and the collection section storage chamber to internally communicate with each other.

15. The method according to claim 1, wherein
the reagent storage chamber serves as a reaction chamber in which the incubation step and the reaction step are performed, contains a mixed reagent of the reagent and the fluorescently-labeled binding molecule, and further comprises a detection region,
the detection region is formed of a member capable of detecting a fluorescence polarization degree in the collection section storage chamber from outside, and
the collection section placed inside the collection section storage chamber is placed inside the reagent storage chamber after the preparation step to bring the mixed reagent in the reagent storage chamber into contact with the collection section and perform the incubation step and the reaction step in parallel.

16. The method according to claim 1, wherein
the reagent storage chamber separately comprises a first reagent storage chamber and a second reagent storage chamber,
the first reagent storage chamber contains the reagent,
the second reagent storage chamber contains the fluorescently-labeled binding molecule,
the first reagent storage chamber and the second reagent storage chamber are separated from the collection section storage chamber,
the first reagent storage chamber serves as a reaction chamber in which the incubation step is performed,
the second reagent storage chamber serves as a reaction chamber in which the reaction step is performed and further comprises a detection region,
the detection region is formed of a member capable of detecting a fluorescence polarization degree in the second reagent storage chamber from outside,
the collection section placed inside the collection section storage chamber is placed inside the first reagent storage chamber after the preparation step to bring the collection section into contact with the reagent in the first reagent storage chamber and perform the incubation step, and
the collection section placed inside the first reagent storage chamber is placed inside the second reagent storage chamber after the incubation step to introduce a bacteria-concentrated reagent after the incubation into the second reagent storage chamber and perform the reaction step.

17. The method according to claim 15, further comprising:
a disinfection step of treating the incubated sample with a disinfectant after the detection step, wherein
the main body further comprises a disinfectant storage chamber that contains the disinfectant,
the reagent storage chamber and the collection section storage chamber are separated from the disinfectant storage chamber, and
the disinfectant storage chamber and the collection section storage chamber internally communicate with each other after the detection step to introduce the disinfectant from the disinfectant storage chamber to the collection section storage chamber and perform the disinfection step.

18. The method according to claim 1, wherein
the reagent is a medium for the target bacterium.

19. The method according to claim 1, wherein
a binding molecule to be bonded to the target bacterium in the fluorescently-labeled binding molecule is at least one selected from the group consisting of an aptamer, a low-molecular-weight compound, a carbohydrate chain, a peptide, a protein, a nucleic acid, virus, and phage.

20. The method according to claim 19, wherein
the protein is an antibody.

21. A bacterial detection tool for use in the method according to claim 1, the bacterial detection tool comprising:
a sample collection tool; and
a main body, wherein
the sample collection tool comprises:
a collection section in which a sample is collected; and
a connection section that is connected to the main body,
the main body comprises:
a reagent storage chamber that contains the reagent and the fluorescently-labeled binding molecule;
a collection section storage chamber that contains the collection section of the sample collection tool; and
a connection section that is connected to the sample collection tool,
the reagent storage chamber and the collection section storage chamber are separated from each other, and
the sample collection tool and the main body are connected to each other at the connection section of the sample collection tool and the connection section of the main body with the collection section of the sample collection tool being placed inside the collection section storage chamber of the main body.
